(19) United States
(12) Patent Application Publication
(10) Pub. No.: US 2014/0182591 A1
(43) Pub. Date: Jul. 3, 2014

(54) PROXIMAL CONTROL VALVE

(71) Applicant: KORE3 INDUSTRIES, LLC, TANGENT, OR (US)

(72) Inventors: RICHARD T. BOONE, SWEET HOME, OR (US); STEWART A. HAMILTON, LEBANON, OR (US)

(73) Assignee: KORE3 INDUSTRIES, LLC, TANGENT, OR (US)

(21) Appl. No.: 14/145,610

(22) Filed: Dec. 31, 2013

Related U.S. Application Data
(60) Provisional application No. 61/747,855, filed on Dec. 31, 2012.

(51) Int. Cl. A61M 16/20; A61M 16/10

(52) U.S. Cl. CPC A61M 16/201; A61M 16/1005
USPC 128/205.24

(57) ABSTRACT

A proximal control valve system for regulating flow of oxygen includes an oxygen inlet attached to an oxygen outlet with an oxygen regulator being positioned therebetween. The oxygen inlet includes a spring plunger assembly that engages with one of a plurality of indents on the gas regulator to enable the flow of oxygen from the oxygen inlet through a desired thru hole in the oxygen regulator to the oxygen outlet. The oxygen regulator is configured to adjust a flow level of the oxygen to a desired level by rotating the oxygen regulator up to 360° about a central axis, allowing the oxygen to flow through the desired thru hole. The system works with a low pressure oxygen source and is positioned proximal to a user such that the user is not required to move to the oxygen source to adjust the flow level of the oxygen.

### RELATED APPLICATION

This application claims priority to provisional patent application U.S. Ser. No. 61/747,855 filed on Dec. 31, 2012, the entire contents of which is herein incorporated by reference.

### BACKGROUND

The embodiments herein relate generally to valves, and more particularly, to a proximal control valve.

Home oxygen dependent users, for many reasons, suffer from acute low blood oxygen levels that can easily be resolved by temporarily increasing oxygen flow. This requires the user to adjust the flow of oxygen coming from the oxygen supply source. Typically, the oxygen flow regulator is located at the oxygen supply source, while the point of use of the oxygen can be a distance of up to 100 feet or more from the oxygen source. This requires the oxygen user to ambulate to the flow source, if able, or the user is dependent on others to increase the oxygen flow or the user must resort to activation of Emergency Medical Services.

Therefore, what is needed is a device that allows a home oxygen user to adjust the gas flow rate proximal to their point of use instead of distally at the gas source.

### SUMMARY

A proximal control valve system for regulating flow of oxygen includes an oxygen inlet attached to an oxygen outlet with an oxygen regulator being positioned therebetween. The oxygen inlet includes a spring plunger assembly that engages with one of a plurality of indents on the gas regulator to enable the flow of oxygen from the oxygen inlet through a desired thru hole in the oxygen regulator to the oxygen outlet. The oxygen regulator is configured to adjust a flow level of the oxygen to a desired level by rotating the oxygen regulator up to 360° about a central axis, allowing the oxygen to flow through the desired thru hole. The system works with a low pressure oxygen source and is positioned proximal to a user such that the user is not required to move to the oxygen source to adjust the flow level of the oxygen.

### BRIEF DESCRIPTION OF THE FIGURES

The detailed description of some embodiments of the invention is made below with reference to the accompanying figures, wherein like numerals represent corresponding parts of the figures.

FIG. 1 is a forward perspective view of one embodiment of the present invention.

FIG. 2 is a schematic rear perspective view of one embodiment of the present invention.

FIG. 3 is an exploded view of one embodiment of the invention.

FIG. 4 is a rear perspective view of one embodiment of the oxygen regulator.

FIG. 5 is a front perspective view of one embodiments of the oxygen regulator.

FIG. 6 is a perspective view of one embodiment of the spring plunger assembly.

FIG. 7 is an exploded view of one embodiment of the spring plunger assembly.

FIG. 8 is a forward perspective view of an embodiment of the present invention.

FIG. 9 is a cross-section view of an embodiment of the invention.

FIG. 10 is a cross-section view of an embodiment of the invention.

FIG. 11 is a cross-section view of an embodiment of the invention.

FIG. 12 is a cross-section view of an embodiment of the invention.

FIG. 13 is a cross-section view of an embodiment of the invention.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The system of the present disclosure may be used to adjust the flow of oxygen proximal to a point of use. This list of possible constituent elements is intended to be exemplary only, and it is not intended that this list be used to limit the system of the present application to just these elements. Persons having ordinary skill in the art relevant to the present disclosure may understand there to be equivalent elements that may be substituted within the present disclosure without changing the essential function or operation of the system.
1. Oxygen Inlet
2. Oxygen Regulator
3. Oxygen Outlet

The various elements of the proximal control valve system for adjusting gas flow of the present disclosure may be related in the following exemplary fashion. It is not intended to limit the scope or nature of the relationships between the various elements and the following examples are presented as illustrative examples only.

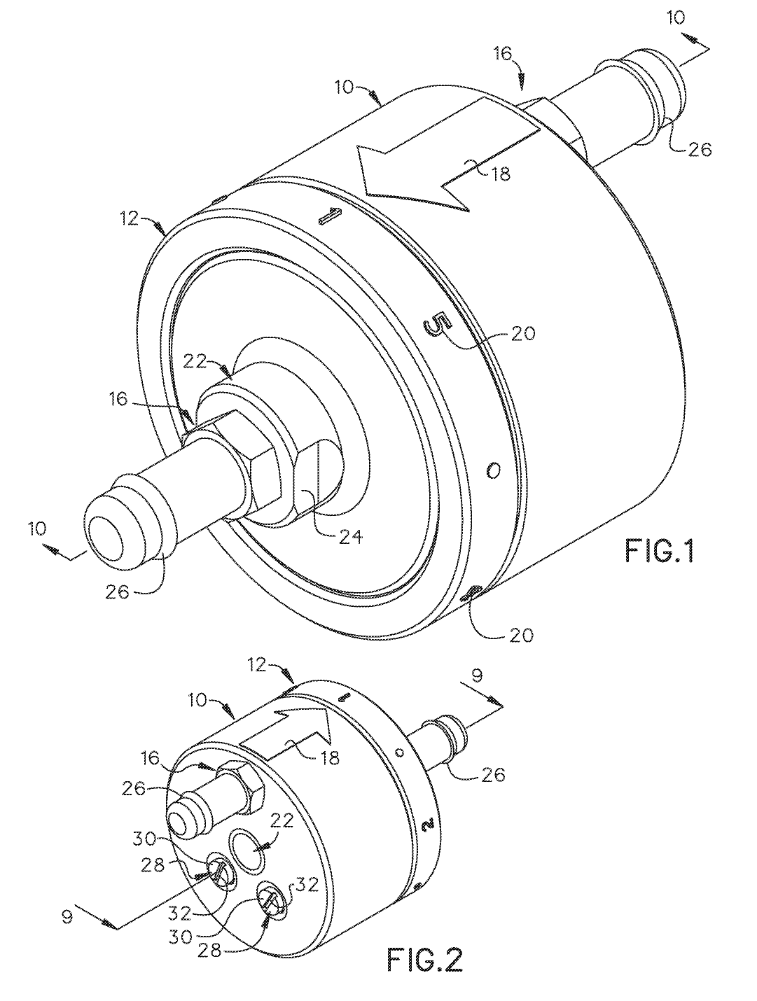

By way of example, and referring to FIGS. 1-3, one embodiment of the present system comprises an oxygen inlet 10 attached to an oxygen outlet 22 with an oxygen regulator 12 being positioned therebetween, wherein the oxygen outlet 22 has a threaded portion that extends through an orifice in the oxygen regulator 12 and screws into a threaded orifice in the oxygen inlet 10. The oxygen inlet 10, the oxygen regulator 12, and the oxygen outlet 22 may be generally disk-shaped and concentrically aligned. In embodiments, and as shown in FIGS. 1-3, 10, and 11, the oxygen inlet 10 may allow the entrance of a gas, such as low pressure oxygen, to enter the system from a gas source. For example, the oxygen inlet 10 may be designed to engage with an exemplary oxygen tube 50, which connects the proximal control valve system to a gas source. The oxygen inlet 10 may engage with the exemplary tube 50 through the use of a threaded screw barbed tube interface barb 26 having a threaded screw barbed tube interface 16. The barb 26 may receive low pressure oxygen, such as oxygen having a flow of from about 1 liter per minute (LPM) to about 5 LPM, from a source, such as a home oxygen concentrator. The threaded screw barbed tube interface 16 may have any suitable diameter and, in embodiments, may have a diameter of 0.125 inches. In embodiments, the gas may then flow through an inlet o-ring 38 and a regulator thru hole 42 to an oxygen outlet channel 52, which extends through the oxygen outlet and a threaded screw barbed tube interface 16 to an exemplary tube 50 for transportation to a user.

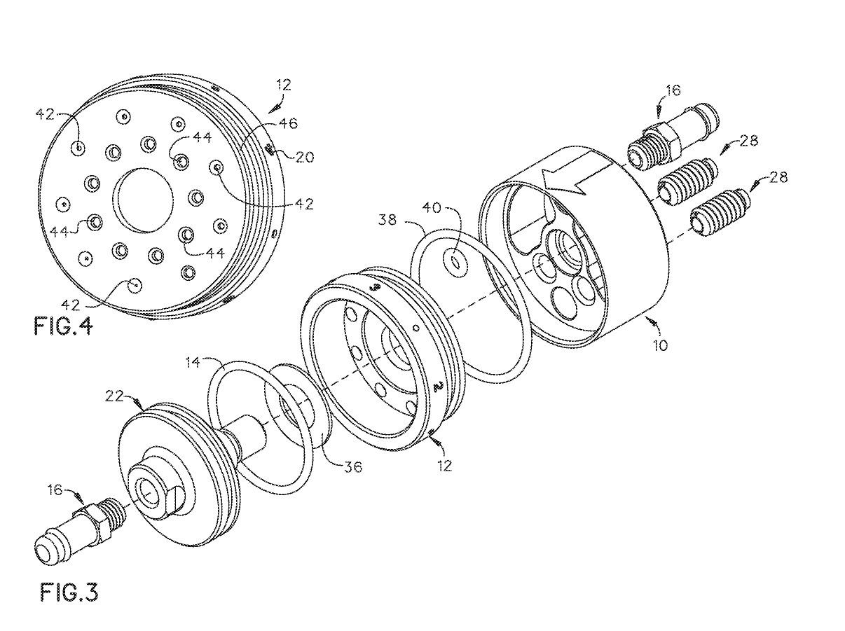

As shown, for example, in FIG. 3, the oxygen regulator 12 may be positioned between the oxygen inlet 10 and the oxygen outlet 22, the oxygen outlet 22 being attached to the oxygen inlet 10 using threads. An oxygen outlet o-ring and a thrust washer may be positioned between the oxygen outlet 22 and the oxygen regulator 12. The oxygen outlet 22 may comprise a threaded screw barbed tube interface 16 designed to engage with an exemplary tube 50, which transports the oxygen from the proximal control valve system to a user.

As shown in FIGS. 2 and 4, the oxygen inlet 10 may comprise a spring plunger assembly 28, which may be configured to interact with an indent 44 located on the oxygen regulator 12. In embodiments, the oxygen inlet 10 comprises a plurality of spring plunger assemblies 28, such as two spring plunger assemblies, and the oxygen regulator 12 comprises a plurality of indents 44, such as nine indents. The oxygen regulator 12 may also comprises a plurality of thru holes 42, each thru hole having a different diameter.

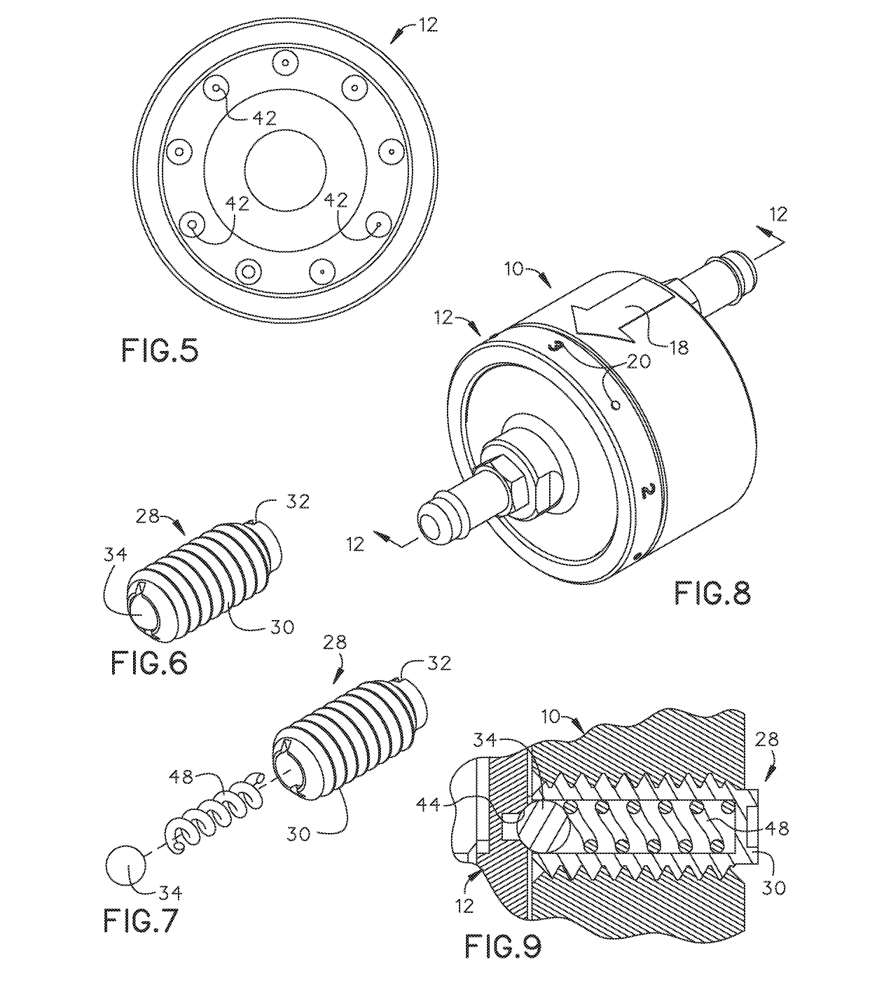

As shown in FIGS. 6, 7, and 9, the spring plunger assembly 28 may comprise a spring plunger shell 30 having a spring plunger shell slot 32 at a distal end thereof. A spring plunger spring 48 may be positioned within the spring plunger shell 30. A spring plunger ball 34 may be positioned at a proximal end of the spring plunger shell 30, the spring plunger ball 34 being positioned to hold the spring plunger spring 48 within in spring plunger shell 30.

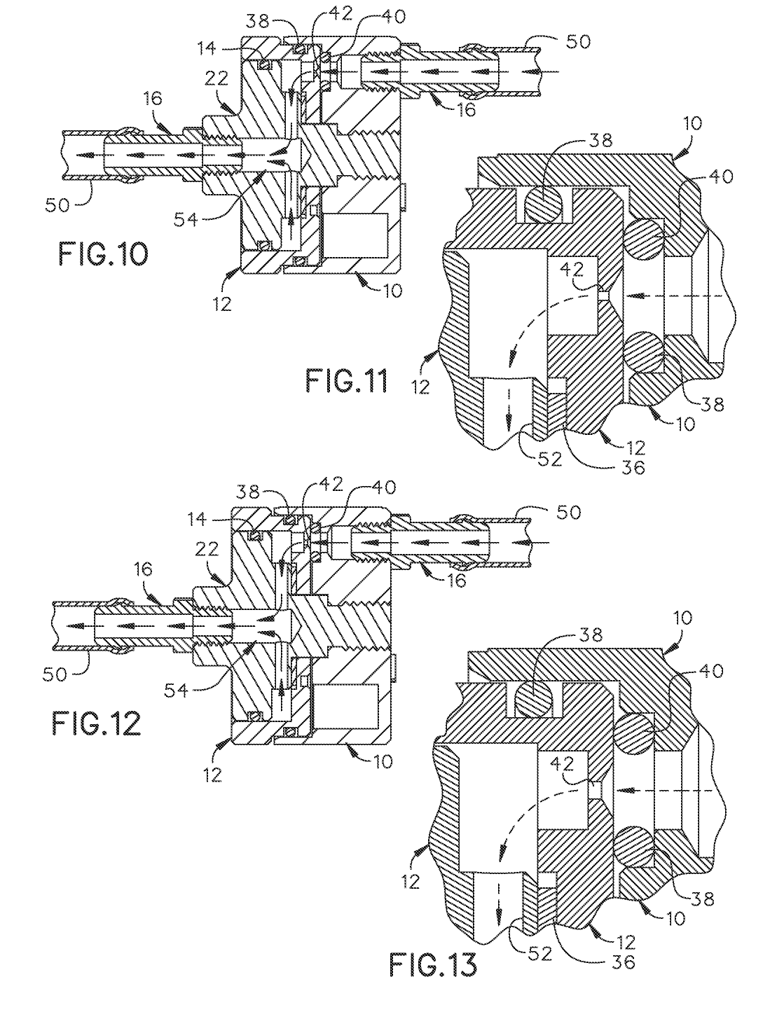

As shown in FIGS. 3-5, the oxygen regulator 12 may comprise a plurality of indents 44 configured to engage with the spring plunger assembly 28, a plurality of regulator thru holes 42, which allow the passage of the desired flow of oxygen from the oxygen inlet 10 to the oxygen outlet 22, and a slot in the regulator 46 for a regulator o-ring 38. A spring plunger assembly 28 may align with any one of the indents 44, which results in the desired thru hole 42 being aligned with the inlet barb 26 and sealed to the inlet o-ring 40, allowing the passage of oxygen therethrough. Thus, gas flow may be regulated depending on which of the indents 44 engages with the spring plunger assembly 28. In other words, gas flow may be regulated by rotating the oxygen regulator 12 to align the indent 44 with the spring plunger assembly 28, the pressure of the spring plunger assembly 28 on the indent 44 resulting in the desired thru hole 42 being positioned to allow gas to flow therethrough. In embodiments, the smaller the diameter of the desired thru hole 42, the lower the flow of the gas; and the larger the diameter, the higher the flow of gas. For example, the thru hole 42 has a larger diameter in FIGS. 12 and 13 than in FIGS. 10 and 11, meaning that the flow level of the oxygen would be greater in the embodiment depicted in FIGS. 12 and 13 than in FIGS. 10 and 11. The gas flow may be controlled by rotating the oxygen regulator 12 up to 360° to a desired flow setting. In embodiments, the oxygen regulator 12 may comprise oxygen regulator level indicators 20 to inform a user of the flow setting of the gas.

In embodiments, the gas may flow through the desired regulator thru hole 42 of the oxygen regulator 12 to the oxygen outlet 22. For example, the gas may flow from the desired regulator thru hole 42 through a chamber to an oxygen outlet channel 52, as shown in FIG. 11. The oxygen outlet channel 52 may extend through a threaded screw barbed tube interface 16 of the oxygen outlet 22, as shown in FIG. 10. The threaded screw barbed tube interface 16 may be designed to engage with an exemplary tube 50, which transports the gas to a user's nasal cannula.

Some embodiments include a thrust washer 36 positioned between the oxygen regulator 12 and the oxygen outlet 22. The thrust washer 36 may function as a pressure plate between the oxygen regulator 12 and the oxygen outlet 22. The thrust washer 36 may be made from a low friction material, such as Delrin.

A user may regulate the flow of oxygen to the desired flow by rotating the oxygen regulator 12 between the oxygen inlet 10 and the oxygen outlet 22, aligning the inlet barb 26 with the desired regulator thru hole 42, which becomes aligned with a channel 52 created by the interface of the oxygen regulator 12 and the oxygen outlet 22. The proximal control valve system may include an oxygen inlet arrow indicator 18 that points to the desired flow level indicator 20. For example, the flow level indicators 20 may be numbered 1-5. In embodiments, the different flow level indicators 20 may correspond to changes in flow in 0.5 LPM increments.

The system may be made of any suitable material, such as machined aluminum or plastic, such as injected molded high density polyethylene plastic.

The system may function as a proximal control valve that is lightweight and ties in line to an existing home oxygen system, such that the system is located near the user. For example, the design may allow the system to rest on a user's chest. The user may perform a one-time adjustment of the flow meter on the home oxygen concentrator. For example, the user may adjust the flow meter on the home oxygen concentrator at any flow rate up to about five liters per minute (LPM). Conventional tubing, such as standard non-crushable oxygen extension tubing, may then be attached to the concentrator's flow meter. The oxygen inlet 10 of the system may then be attached to the end of the extension tubing, and tubing, such as a one foot standard nasal cannula, may be attached to the oxygen outlet 22. The user may then set the system to the desired flow rate. If the user feels short of breath or experiences chest pain, then the user may increase the oxygen flow, for example by an additional one to two liters per minute in half liter per minute increments, by rotating the system. The increased flow rate may continue until the user or a care provider decreases the flow rate. The user or care provider may check the pulse oximetry level to ensure that the user achieves adequate tissue oxygenation.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A proximal control valve system for regulating flow of gas from a gas source to a user, the system being positioned proximal to a user, the system comprising:
a gas inlet attached to a gas outlet with a gas regulator being positioned between the gas inlet and the gas outlet,
the gas inlet being configured to transport gas from a gas source to the gas regulator, the gas inlet comprising a spring plunger assembly configured to engage with one of a plurality of indents on the gas regulator resulting in the gas to flow through a desired thru hole on the gas regulator,
the gas regulator being configured to adjust a flow level of the gas to a desired level and to transport the gas from the gas inlet to the gas outlet, and
wherein the system is configured to be positioned proximal to a user such that the user is not required to move to the gas source to adjust the flow level of the gas.

2. The system of claim 1, wherein the gas outlet has a threaded portion that extends through an orifice of the gas regulator and screws into a threaded orifice of the gas inlet, the gas regulator being capable of rotating up to 360° about the threaded portion of the gas outlet.

3. The system of claim 1, wherein the gas inlet comprises a threaded screw barb configured to engage with a tube extending from the gas source, the threaded screw barb being generally cylindrical shaped and having an inner orifice that allows for the flow of gas therethrough,
the threaded screw barb being configured to engage with a regulator thru hole on the oxygen regulator, which enables the gas to flow from the tube through the threaded screw barb and the regulator thru hole to a gas outlet channel of the gas outlet, which is configured to engage with a second tube for transportation of the gas to a user.

4. The system of claim 1, wherein the gas inlet comprises two spring plunger assemblies that are configured to engage with two of the plurality of indents on the gas regulator thereby aligning the desired thru hole with an inlet o-ring, a pressure exerted by the spring plunger assembly onto the gas regulator sealing the desired thru hole to the inlet o-ring allowing gas to pass from the gas inlet through the gas regulator to the gas outlet.

5. The system of claim 2, wherein:
the gas regulator comprises a plurality of indents and a plurality of regulator thru holes;
when the gas regulator is rotated about the threaded portion of the gas outlet, a first portion of a plurality of spring plunger assemblies are engaged with the plurality of indents on the gas regulator creating seals; and
one regulator thru hole of the plurality of thru holes is sealed with an inlet o-ring, allowing the gas to flow therethrough and to the gas outlet.

6. The system of claim 5, wherein the flow level of the gas is dependent on which thru hole is sealed, the system being configured to adjust the flow level of the gas in predetermined increments.

7. A proximal control valve system for regulating flow of oxygen from an oxygen source to a user, the system being positioned proximal to a user, the system comprising:
an oxygen inlet attached to an oxygen outlet with an oxygen regulator being positioned between the oxygen inlet and the oxygen outlet;
the oxygen inlet being configured to transport oxygen from an oxygen source to an oxygen regulator, the oxygen inlet comprising a spring plunger assembly configured to engage with one of a plurality of indents on the oxygen regulator resulting in the oxygen to flow through a desired thru hole in the oxygen regulator to the oxygen outlet;
the oxygen regulator being configured to adjust a flow level of the oxygen to a desired level by rotating the oxygen regulator up to 360° about a central axis, wherein:
the system is configured to be positioned proximal to a user such that the user is not required to move to the oxygen source to adjust the flow level of the oxygen; and
the system is configured to work with a low pressure oxygen source.

8. The system of claim 7, wherein:
the low pressure oxygen source has a flow of from about 1 liter per minute (LPM) to about 5 LPM; and
the system is configured to adjust the flow level of the gas in predetermined increments.

9. The system of claim 8, wherein the predetermined increments are 0.5 LPM increments.

10. The system of claim 8, wherein the oxygen regulator comprises an oxygen regulator level indicator that is capable of communicating the flow level of the oxygen to the user.
